(19) United States
(12) Patent Application Publication
(10) Pub. No.: US 2011/0015491 A1
(43) Pub. Date: Jan. 20, 2011

(54) SURGICAL ACCESS DEVICE WITH MOVEABLE DEVICE PORT

(76) Inventors: Sundaram Ravikumar, Briarcliff Manor, NY (US); Allan Alward, Shelton, CT (US)

Correspondence Address: EDWARDS ANGELL PALMER & DODGE LLP, P.O. BOX 55874, BOSTON, MA, 02205, US

(21) Appl. No.: 12/504,605

(22) Filed: Jul. 16, 2009

(51) Int. Cl. A61B 1/32

(52) U.S. Cl. 600/233

(57) ABSTRACT

A surgical access device for use in laparoscopic surgical procedures includes a housing portion and at least one moveable device port. The housing portion has an axial bore extending therethrough, defining a central axis of the surgical access device. The at least one moveable device port is moveable about an annular path corresponding to an annular seal member. Each moveable device port defines a lumen extending therethrough, and each lumen is substantially parallel to the central axis of the surgical access device.

### FIELD OF THE INVENTION

The present invention relates to surgical access devices, such as those used in laparoscopic surgical procedures, typically within the abdominal cavity of the patient. Particularly, the present invention is directed to a surgical access device having at least one device port that is moveable with respect to a housing of the access device.

### DESCRIPTION OF RELATED ART

A variety of surgical access devices are known in the art for maintaining an operative path through an anatomical structure, particularly a patient's abdominal wall, to facilitate insertion of surgical instruments during minimally-invasive (laparoscopic) surgical procedures. Typically, one or more instruments are inserted through such access devices into the peritoneal space, which is insufflated during a procedure, typically with an inert gas such as carbon dioxide. Such access devices maintain the incision opening and minimize trauma through repeated insertion and manipulation of instruments, removal of specimens, and the like. Typically, such access devices are relatively small in size—between about 1.0 and 2.0 centimeters in diameter. Because of size limitations, it is often necessary to utilize multiple access ports in a single procedure, distributed across the abdomen, for example.

Typical access ports are provided with mechanical seals, usually formed from a resilient material. Due to the need for simultaneous use of different instruments, it is often desirable to insert multiple instruments through a single access port. However, with a conventional arrangement, simultaneous insertion of multiple instruments suffers from mutual interference between adjacent instruments and loss of insufflation pressure and gas—loss of the pneumoperitoneum. Accordingly, with previous devices it has proven difficult to achieve desirable results when using multiple instruments through a single access device.

Devices constructed in accordance with the present invention are particularly adapted to use with multiple instruments. The subject devices maintain sealed access through the anatomical structure, such as the abdominal wall, and advantageously stabilize and maintain separation between instruments used simultaneously, avoiding the disadvantages of prior devices.

The subject devices are particularly well suited for use in single-incision laparoscopic surgery or “SILS” procedures. In such procedures, a single incision is formed for access to the operative space, such as through the abdominal wall and into the abdominal cavity. Such procedures advantageously result in minimal to no visible scarring of the patient, particularly when made through the umbilicus. Performance of such procedures suffers from various disadvantages, with adjacent instruments interfering with one another, or promoting loss of insufflation gas. Devices in accordance with the present invention, however, maintain sealed and stabilized access of multiple instruments simultaneously through a common access device, inserted though a single incision.

### SUMMARY OF THE INVENTION

The purpose and advantages of the present invention will be set forth in and apparent from the description that follows. Additional advantages of the invention will be realized and attained by the devices, and the related methods particularly pointed out in the written description and drawings hereof.

To achieve the above-mentioned and additional advantages, in accordance with one aspect of the invention, a surgical access device for use in laparoscopic surgical procedures is provided, having a housing portion and at least one moveable device port. The housing portion has an axial bore extending therethrough, defining a central axis of the surgical access device. The at least one moveable device port is moveable about an annular path corresponding to an annular seal member. Each moveable device port defines a lumen extending therethrough. Each lumen is substantially parallel to the central axis of the surgical access device.

In accordance with an alternate aspect of the invention, a surgical access device can be configured with a linear path, or alternatively, a path having another shape. In accordance with such aspects, the seal member includes a corresponding shape to maintain sealed access through the surgical access device.

The optional features described herein in connection with a particular embodiment can be incorporated specifically as described, or alternatively such features can be combined in combinations not specifically set forth herein, still in keeping with the spirit of the invention.

The subject surgical access devices can include one substantially axially central stationary device port, or alternatively, more than one such port, held in the housing portion. The stationary device port or ports are preferably restrained from lateral and axial movement, relative to the central axis. Such ports can be completely stationary, or alternatively can be rotatable about a central axis thereof, or alternatively or additionally rotatable about other axes, if desired.

One of the moveable device ports, or more if a plurality thereof is provided, preferably includes a seal member held within a moveable housing. As set forth above, the path, in accordance with one aspect, is annular in configuration. Alternatively, such paths can be provided in alternative shapes, including but not limited to linear, undulating, circular, ovoid, oblong, and so on. The seal member of the moveable device port can be embodied to include a duckbill-type sealing element, configured to seal the lumen of the moveable device port in the absence of an instrument inserted therethrough. Additionally, if so embodied, the seal member of the moveable device port can include an annular seal configured to sealably engage an outer surface of an instrument when inserted therethrough.

In accordance with the invention, a single moveable device port can be provided, or alternatively a plurality of moveable device ports can be provided, such as two, three, four, five, six, seven, eight, nine, ten or more in number.

In accordance with the invention, the circular path can be an annular channel defined by the housing. The annular channel can be configured to restrain the moveable device port from moving in any direction other than along the circular path. Particularly, the moveable device ports can be restrained from radial movement beyond a predetermined region. Additionally or alternatively, the device ports can be restrained from axial movement beyond a predetermined region.

The annular channel is defined within the housing by radially outer and inner channel walls and axially upper and lower channel walls. The axially upper and lower channel walls can be configured to only partially extend radially toward a centerline of the annular channel, permitting passage of instruments through a moveable device port lumen and through the upper and lower channel walls. The annular channel can include a plate formed of a lubricious material on the axially lower channel wall, facilitating movement of the at least one moveable device port. The annular seal member can be compressed by and at least partly positionally secured by the plate.

The annular seal member can include two sealing portions or leaves, extending toward one another about the annular path, and resiliently mutually contacting one another to inhibit flow of insufflation gas though the seal member.

In accordance with a further aspect of the invention, surgical access devices in accordance with the invention can include a body tube extending from a lower end of the housing portion. The body tube, if provided, defines a central lumen extending axially therethrough. At least one engagement feature can optionally be provided on the body tube to facilitate engagement of the surgical access device with an anatomical structure of a patient, such as a portion of the abdominal wall of the patient.

If provided, the at least one engagement feature can be a substantially annular engagement member extending radially outwardly from an outer circumferential wall of the body tube. The annular engagement member can be inflatable with a fluid to expand the volume of the engagement member to promote engagement with the anatomical structure. Accordingly, the annular engagement member can be in communication with an insufflation connection on the surgical access device. A multi-way valve can be provided, interposed between the insufflation connection, an insufflation output into the surgical access device and into at least one engagement member. Accordingly, the engagement members can be inflatable with a gas such as air, or carbon dioxide, or alternatively can be embodied for inflation with a liquid, such as saline.

Surgical access devices can be provided with an insufflation port in fluid communication with the lumen, arranged below the annular seal member, for example, adapted and configured for providing insufflation gas to an operative cavity.

If desired, at least one anchor point on the housing portion can be provided, configured and adapted for engagement with a suture material for tethering to an anatomical structure of a patient. Such anatomical structure can be a portion of the abdominal wall of the patient, for example.

Further, in accordance with another aspect, the invention includes a method of accessing a surgical operative space, comprising the steps of forming an incision through an anatomical structure and inserting a surgical access device through the incision, where the surgical access device has a housing portion having an axial bore extending therethrough, defining a central axis of the surgical access device, and at least one moveable device port, moveable about an annular path corresponding to an annular seal member, each moveable device port defining a lumen extending therethrough, each lumen being substantially parallel to the central axis of the surgical access device. In accordance with this aspect, the operative space can be a patient's abdominal cavity and the anatomical structure can be the patient's abdominal wall. Alternatively, the operative space can be a patient's thoracic cavity, or other area of the body.

It is to be understood that both the foregoing general description and the following detailed description are exemplary and are intended to provide further explanation of the invention.

### BRIEF DESCRIPTION OF THE DRAWINGS

The accompanying drawings, which are incorporated in and constitute part of this specification, are included to illustrate and provide a further understanding of the invention. Together with the description, the drawings serve to explain the principles of the invention, wherein:

FIG. 1 is a top isometric view of a surgical access device constructed in accordance with the invention, with a housing and body tube shown in a separated condition;

FIG. 2 is a bottom isometric view of the surgical access device of FIG. 1, with the housing and body tube shown in a separated condition;

FIG. 3 is a top isometric view of a housing portion of the surgical access device of FIG. 1;

FIG. 4 is a top isometric view of a housing portion of the surgical access device of FIG. 1, shown with an upper portion of the housing removed;

FIG. 5 is an exploded top isometric view of a housing of the surgical access device of FIG. 1, showing internal components of the housing;

FIG. 6 is an exploded bottom isometric view of a top portion of the housing of the surgical access device of FIG. 1;

FIG. 7 is an exploded view of a moveable device port of the embodiment of FIG. 1;

FIG. 8 is a cross-sectional view of the housing of the surgical access device of FIG. 1, taken along line 8-8 of FIG. 1;

FIG. 9 is a perspective view with a partial cutaway of an abdominal wall, with the surgical access device of FIG. 1 inserted therethrough, and surgical instruments shown in conjunction with the surgical access device;

FIG. 10 is a perspective view similar to that of FIG. 10, illustrating the moveable nature of the moveable access ports with respect to the housing;

FIG. 11 is a cross-sectional view of the housing of the surgical access device of FIG. 1, taken along line 11-11 of FIG. 10;

FIG. 12 is a perspective view of an exemplary surgical access device in accordance with the invention showing a threaded connection between housing and body tube portions thereof, having a male thread on the housing portion;

FIG. 13 is a perspective view of a further exemplary embodiment of a surgical access device in accordance with the invention, in which a mechanical interlock feature is provided between the housing and body portions thereof;

FIG. 14 is a top isometric view of a surgical access device in accordance with a further aspect of the invention, having inflatable anchor elements provided on a body tube thereof;

FIG. 15 is a perspective view with a partial cutaway of an abdominal wall, with the surgical access device of FIG. 14, with the anchor elements inflated;

FIG. 16 is a cross-sectional view of the surgical access device of FIG. 14, taken along line 16-16; and

FIG. 17 is a top isometric view of a surgical access device in accordance with the invention, having an engagement-promoting surface treatment provided thereon.

### DETAILED DESCRIPTION OF THE PREFERRED EMBODIMENT

Reference will now be made in detail to the presently preferred embodiments of the invention, examples of which are illustrated in the accompanying drawings.

As set forth hereinabove, the devices and related methods presented herein are particularly suited for laparoscopic procedures, in-particular for single-incision laparoscopic surgeries, or “SILS.” The subject devices provide various advantages, including but not limited to sealed simultaneous insertion of multiple instruments, as well as support and guidance of such instruments. Further, the guidance of such support is adjustable when inserted though a moveable device port.

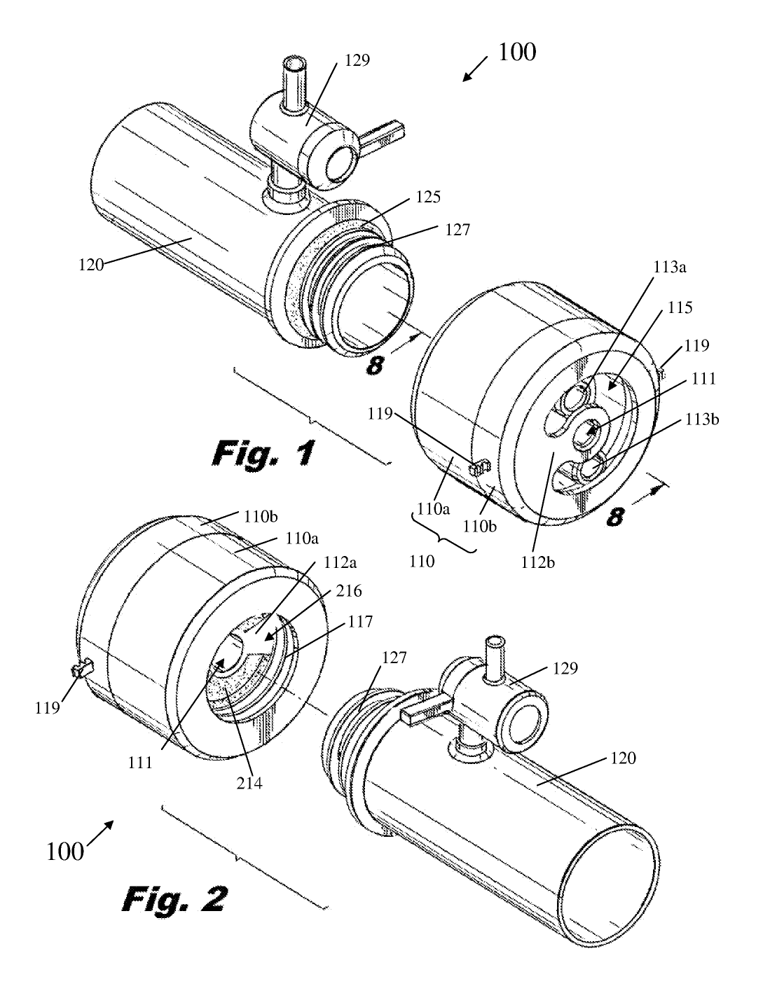

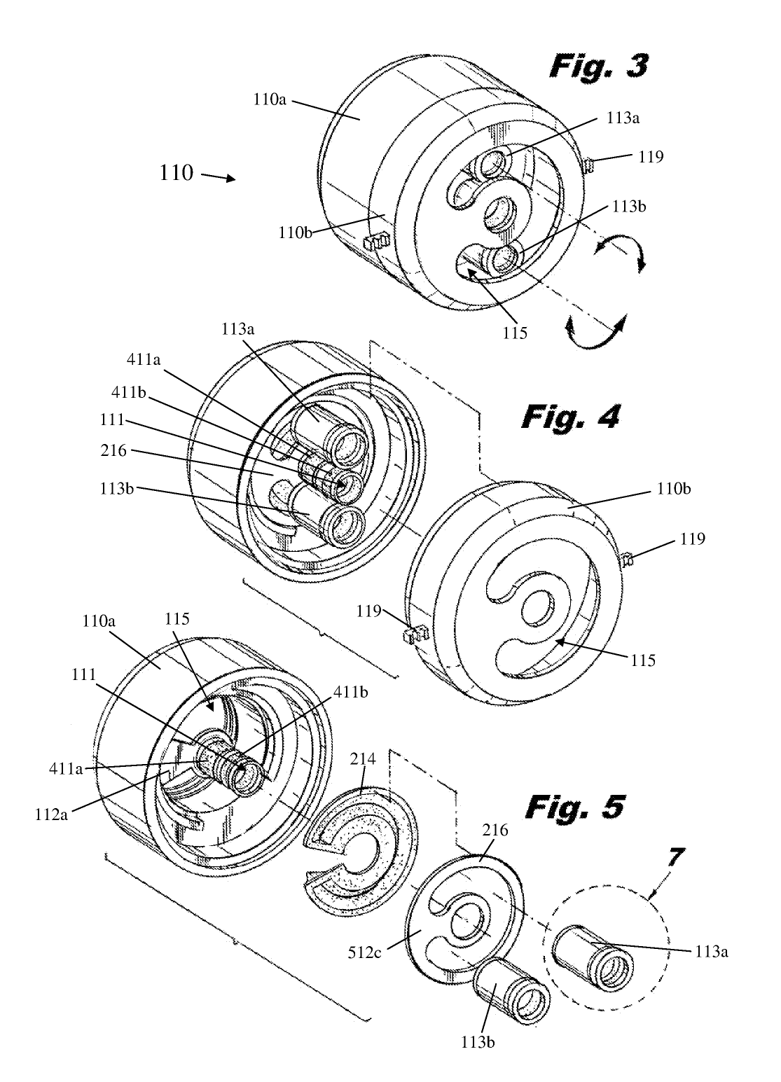

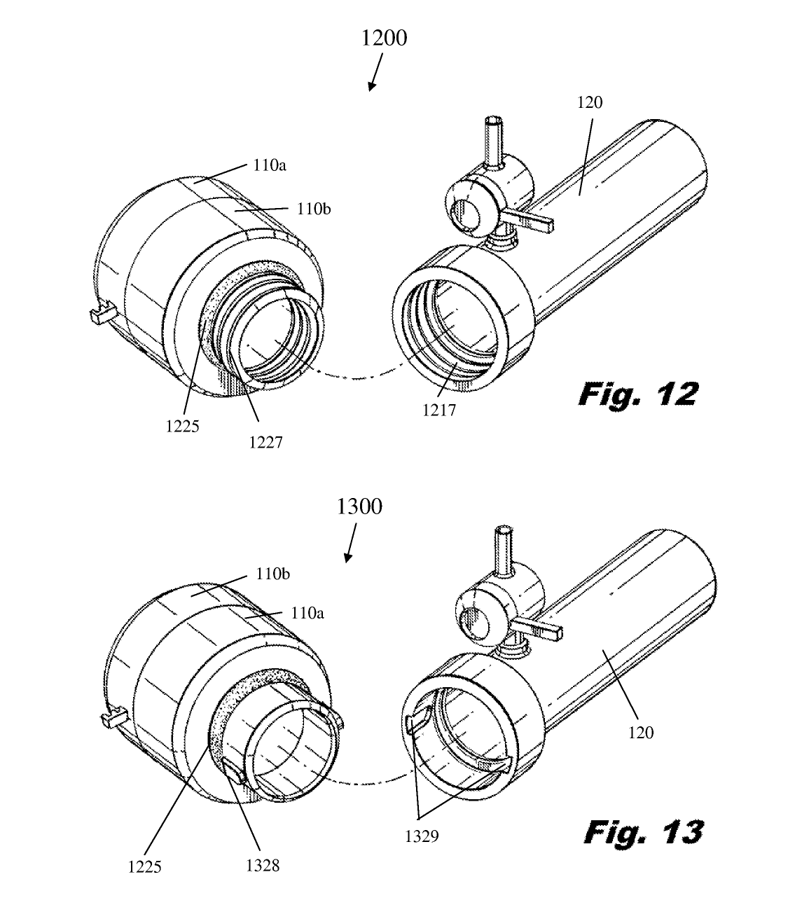

FIGS. 1-11 illustrate various aspects of a surgical access device in accordance with the present invention. FIGS. 1 and 2 illustrate, respectively, top and bottom isometric views of a surgical access device constructed in accordance with the invention, which is designated generally by reference number 100. A housing 110 and body tube 120 are illustrated as separate, connectable elements, and an optional seal 125 is provided for sealing therebetween. In the illustrated embodiment, the housing 110 includes a female threaded portion 117, and the body tube 120 includes a male threaded portion 127. It is to be understood that in this and other embodiments, the configuration of the connection between the housing 110 and the body tube 110 can be configured differently, as discussed below in connection with FIGS. 12 and 13, and alternatively that the housing 110 and the body tube 120 can be formed integrally, or otherwise provided unitarily.

The housing 110 includes a lower portion 110a, and an upper portion 110b. The upper 110b and lower 110a portions can be formed separately and joined by any suitable technique, including but not limited to a threaded connection, mechanical interlock, such as a bayonet-type lock, press or friction fitting, adhesives, heat, solvent or ultrasonic welding, or the like.

The housing 110 and other elements of surgical access devices in accordance with the invention can be formed of polymeric materials, such as plastics, composites, such as fiber-reinforced plastics, or other materials, for example. Seal members described herein can be formed of any suitable materials, such as polymeric materials including elastomers and composites containing elastomers, or other suitable materials, such as silicone, for example. Surfaces that are preferably easily slideable with respect to others can be formed of a low-friction material such as polytetrafluoroethylene, or the like. The housing and other components can be formed from acrylic materials, or the like. Processes for manufacturing the parts can include, without limitation, injection molding, machining vacuum forming of composite materials, or other techniques.

Within the housing 110 are held one or more moveable device ports 113. As illustrated, two such moveable device ports 113a, 113b are provided and held within the housing, as will be describe in more detail below. The housing optionally includes a central device port 111. The central device port 111 is preferably constrained from movement with respect to the housing 110. In accordance with an alternate aspect, lateral and axial movement are substantially inhibited, while rotation relative to a longitudinal axis of the surgical access device 100 is permitted. Alternatively still, the surgical access device 110 can be configured to permit the central device port 111 to move radially, such as along a linear channel (not illustrated), formed in the central device port supports 112a, 112b. Further, multiple central and/or stationary device ports can be provided, combined with moveable ports, as will be appreciated, and which aspect is within the scope of the invention.

With reference to FIGS. 1-5, there is illustrated an annular seal member 214, which corresponds to an annular channel 115 within which the moveable device ports 113a, 113b are permitted to translate and rotate, the configuration and operation of which will be discussed in more detail below. A seal compression plate 216 is provided between the seal member 214 and the annular channel 115, and facilitates movement of the moveable device ports 113. In accordance with one aspect, the compression plate 216 is formed from a lubricious material to reduce excess frictional forces during movement of the moveable device ports 113. As illustrated, a support 112a is provided in connection with the lower housing portion 110a. In conjunction with the support 112b of the upper housing portion 110b, the lower support 112a helps support the central device port 111. A corresponding support 512c is provided in the compression plate 216.

The annular seal member 214, which can be embodied in other configurations, such as linear configurations, permits repositioning or translation of an instrument therethrough, while maintaining a sealed closure across the remainder of the seal member 214. As seen in FIG. 5, the seal member includes two arcuate leaf portions extending toward a center portion thereof, and downwardly (away from the viewer in FIG. 5). As illustrated, the body of the seal member 214 is annular in configuration with an angular discontinuity, the leaf portions thereof terminating at a molded end wall. Alternatively, the member can be continuous, with at least a continuous upper flange portion, for example. Alternatively still, the member 214 can be provided in multiple parts, such as in separate inner and outer annular elements, that are later joined and maintained in position by the respective elements within the housing.

The annular seal member 214 can be formed by molding, such as injection molding, or another suitable process, such as a vacuum lamination process, for example. Materials suitable for construction of the seal member can include, for example, elastomers or composites such as fiber-reinforced polymers, for example. The seal member 214 can be provided with features such as gussets or stays to prevent inversion of the seal under high insufflation pressures.

Further, in the illustrated embodiment, as well as in other embodiments discussed hereinbelow, a valve 129 is provided for insufflation of the operative space, such as the abdominal cavity, during a procedure. A fluid conduit is connected to the valve 129 from a surgical insufflator, for example. The valve 129 is then opened to permit entry of insufflation gas, rotated to release insufflation gas to the surroundings, or as discussed in more detail below, to inflate anchor elements for securing the position of the surgical access device.

Further optional features that can advantageously be applied to devices in accordance with the invention include suture tie-down points or anchor points 119 provided in connection with the housing 110.

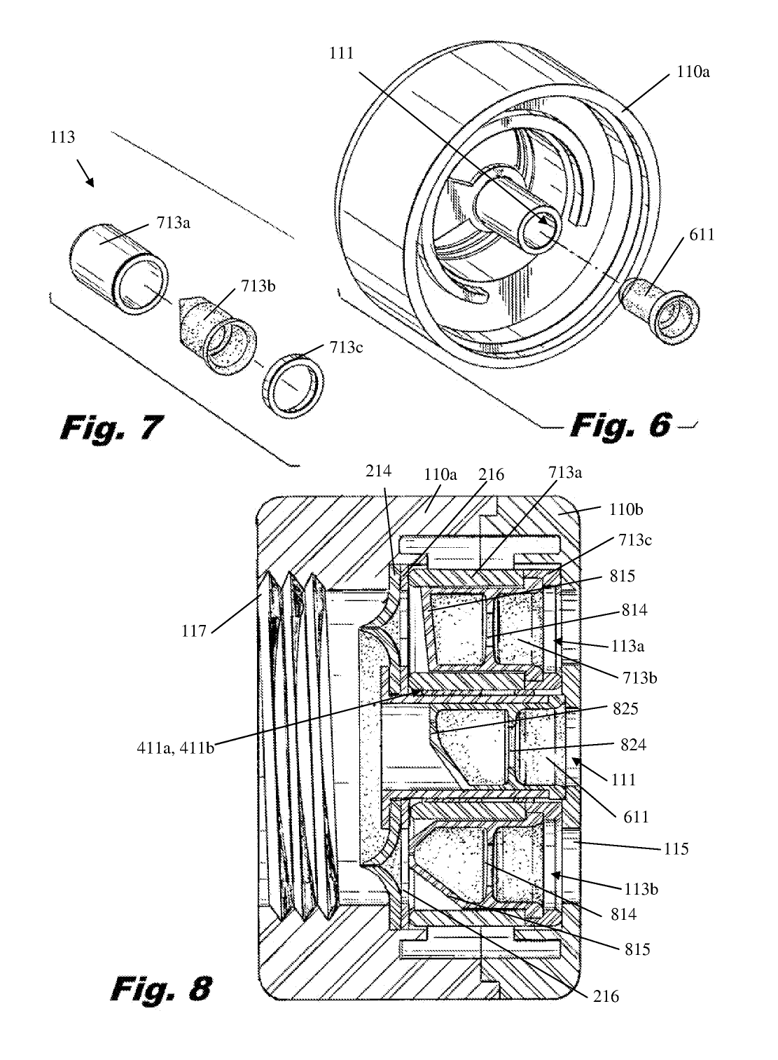

With reference to FIGS. 6-8, the housing 110 has an axial bore extending therethrough, within which the moveable device ports 113 and central device port 111 are held, and which defines a central axis of the surgical access device 100. Moreover, each moveable device port 113a, 113b defines a lumen extending therethrough.

As seen in FIGS. 4, 5 and 8, one or more central radial bushings 411a, 411b can be provided about the central device port 111, between the central device port and the moveable device ports 413a, 413b. The central radial bushings 411a, 411b can be configured to maintain slight frictional engagement between the moveable device ports 413a, 413b. A small amount of friction for maintaining the position of the moveable device ports 413 when not in use is desirable, however it is not desirable that such frictional forces exceed a value where it would become difficult or promote fatigue for a surgeon to move the ports 413.

FIG. 6 is a partial exploded view illustrating a central seal member 611 of the central device port 111 removed from a housing thereof. As illustrated the housing of the central device port 111 is integrally formed with the lower housing portion 110a, but may be formed separately in alternate embodiments.

FIG. 7 is an exploded view of a moveable device port 113 in accordance with the invention, illustrating a lower housing portion 713a, a seal member 713b and an upper housing portion 713c thereof. In the illustrated embodiment, as seen in the cross-sectional view of FIG. 8, the lower housing portion 713a and the upper housing portion 713c are mutually connected and secure the seal member 713b thereto. The components of the moveable device port can be connected in any suitable manner, including mechanical interlock, a threaded connection, adhesive, solvent, heat or ultrasonic welding, or the like.

With reference to FIG. 8, the seal member 713b of the moveable device port 113 can be embodied to include a duckbill-type sealing element 815, configured to seal the lumen of the moveable device port 113 in the absence of an instrument inserted therethrough. Additionally, as illustrated, the seal member 713b of the moveable device port 113 can include an annular seal 814, which is configured to sealably engage an outer surface of an instrument when inserted therethrough. Accordingly, the seal member 713b can be provided as a unit or in separate elements, each of the annular seal 814 and duckbill-type sealing element 815 being formed separately.

Similarly, with reference to FIG. 8, the seal member 611 of the central device port 111 can be provided with both a duckbill-type seal 825, configured to seal the lumen of the central device port 111 in the absence of an instrument inserted therethrough and an annular seal 824, configured to sealably engage an outer surface of an instrument when inserted therethrough.

As best seen in the cross-sectional view of FIG. 8, the annular channel 115 is defined within the housing 110 by radially outer and inner channel walls and axially upper and lower channel walls. As mentioned above, the inner channel wall, as illustrated, is common with the wall defining the central device port 111. The axially upper and lower channel walls can be configured to only partially extend radially toward a centerline of the annular channel, leaving a pathway for access through the annular channel 115, thereby permitting passage of instruments through a lumen of the moveable device ports 113, and through the upper and lower channel walls.

As mentioned above, a compression plate 216, preferably formed of a relatively low friction material, such as a lubricious material, defines the face of the axially lower channel wall and facilitates movement of the moveable device ports 113. The annular seal member 214, is held below the compression plate 216, between the plate 216 and the lower housing portion 110a.

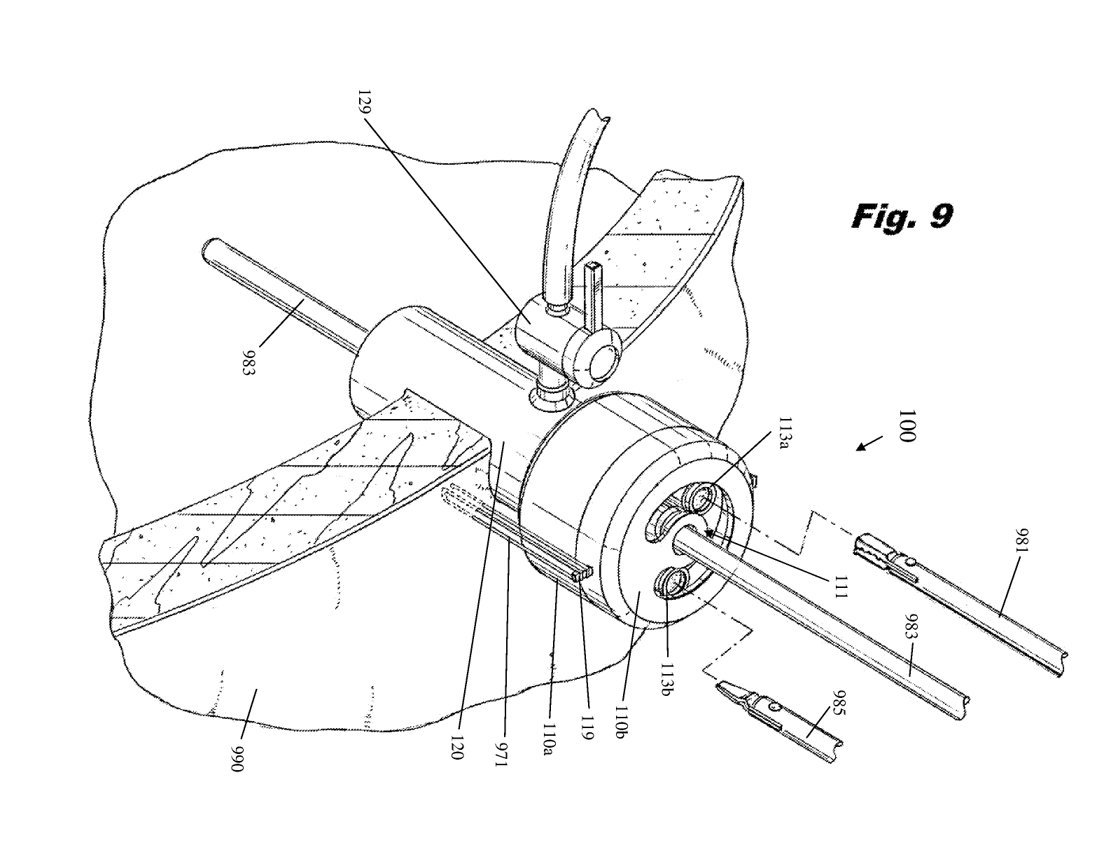

FIGS. 9-11 illustrate the surgical access device 100 in conjunction with multiple surgical instruments. FIG. 9 illustrates a single surgical instrument 983 inserted through the central device port 111, and two instruments 981, 985 respectively positioned for insertion through the moveable device ports 113a, 113b. Also, shown in FIG. 9 are sutures 973 secured to anchor points 119, for stabilizing the surgical access device 110 to the abdominal wall 990.

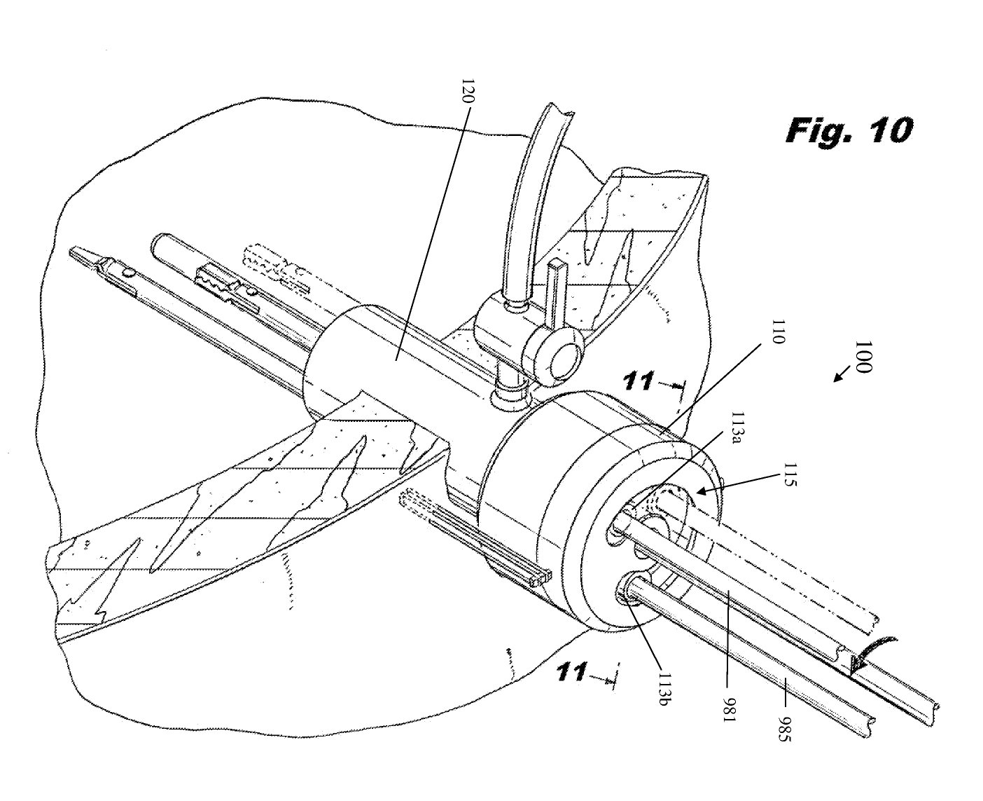

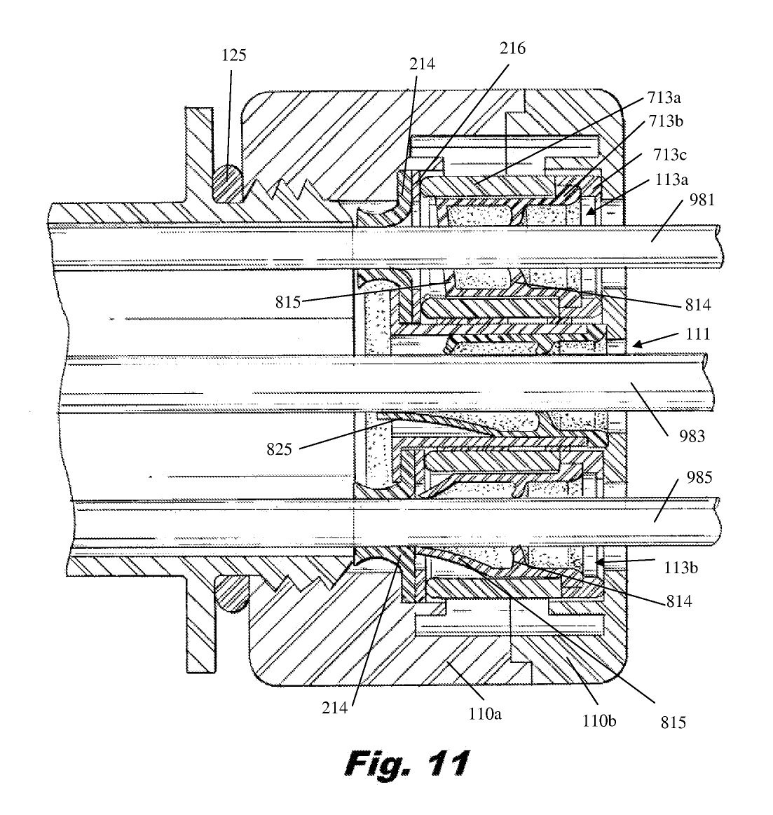

FIG. 10 illustrates the configuration of FIG. 10, wherein one instrument 981 and its respective device port 113a are translated along the channel 115 to a second position, angularly displaced from the initial position, which is illustrated in broken line.

FIG. 11 is a cross-sectional view taken across line 11-11 of FIG. 10, with the instrument 981 and device port 113a shown in their initial position. In this arrangement, surgical instruments 918, 983 and 985 are inserted through the device ports 111, 113a and 113b. As can be seen, the annular seal member 214 flexes to permit passage of the instruments therethrough, as compared to the closed position of FIG. 8, in which the two opposed valve members are in mutual contact to prevent loss of insufflation gas.

The cross-sectional view of FIG. 11 illustrates the duckbill-type seal members 814, 815 opened to permit passage of the instruments therethrough. The annular seal members 814, 824 seal against the shafts of the surgical instruments to prevent escape of any insufflation gasses that may pass through the annular seal member 214 and the duckbill-type seal member 814, 815.

Moreover, each of the moveable device ports 113 can include a seal element in connection therewith to further seal between the device port 113 and the annular seal member 214, such as may be necessary when an instrument is inserted therethrough. When an instrument is inserted through the annular seal member 214 so-called “cat-eyeing” may occur, and may result in small amounts of gas loss. Accordingly, a seal member filling the space between the moveable device port 113 and the annular seal member 214 may be desirable. Such additional seal member can be embodied as a skirt formed of a resilient material and shaped to interface with the arcuately concave surface of the upper face of the annular seal member 214, for example.

FIG. 12 is a perspective view of an exemplary surgical access device 1200 in accordance with the invention, having a threaded connection with male threads 1227 on the housing portion 110 and female threads on the body tube portion 120. A seal 1225 can be provided for sealing between the housing 110 and the body tube 120.

FIG. 13 is a perspective view of a further exemplary embodiment of a surgical access device 1300 in accordance with the invention, in which a mechanical interlock feature having male protrusions 1328 and female recesses 1329 is provided for connection between the housing 110 and body tube 120 portions thereof. Such mechanical interlock can include any suitable features, such as snap fit, friction fit, bayonet-type locking configurations or the like.

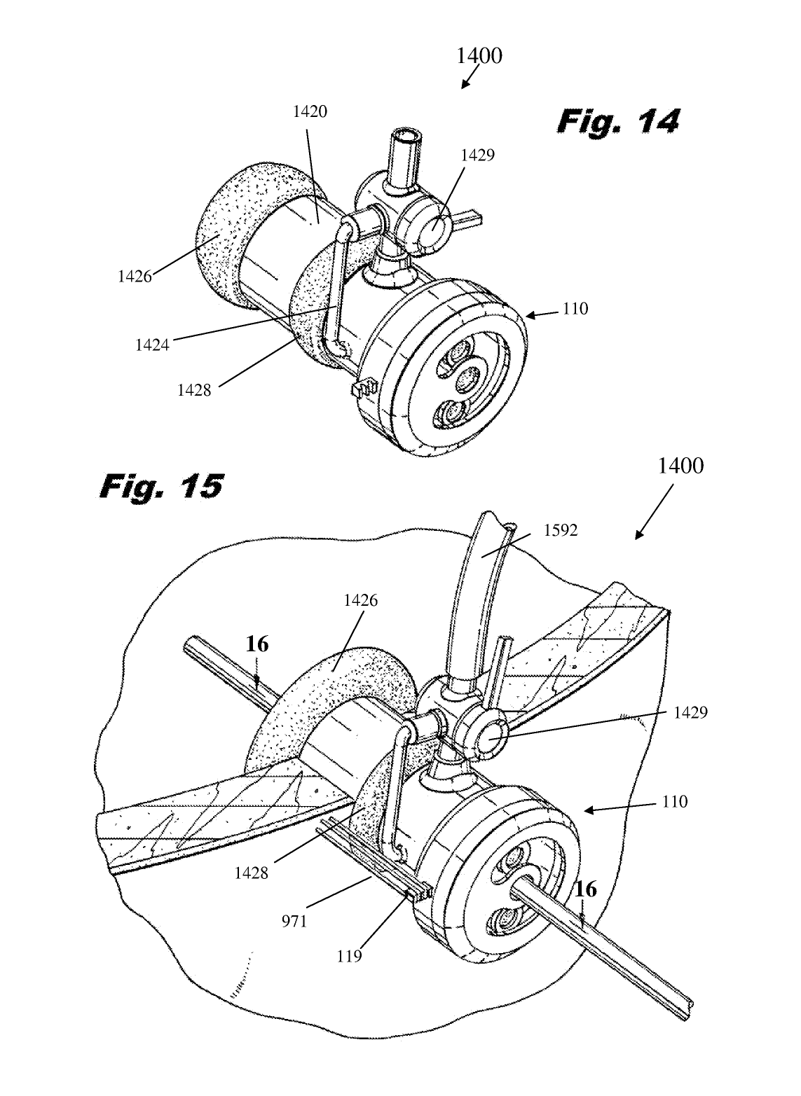

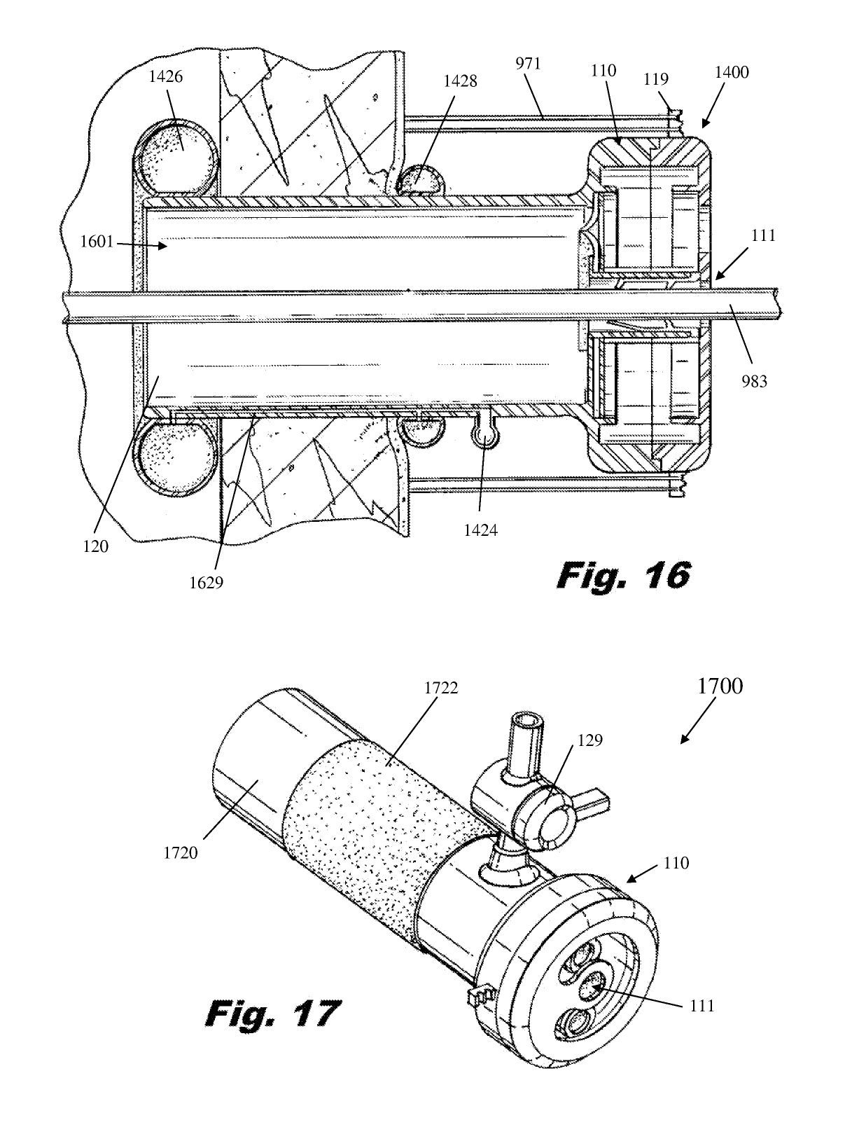

FIGS. 14-16 illustrate a surgical access device 1400 in accordance with a further aspect of the invention, having inflatable anchor elements 1426, 1428 provided on a body tube 1420 thereof. FIG. 14 is a top isometric view of the access device 1400, FIG. 15 is a perspective view with a partial cutaway of an abdominal wall, showing the surgical access device 1400 with the anchor elements inflated, and a fill tube 1592 and FIG. 16 is a cross-sectional view of the surgical access device 1400 taken along line 16-16. The anchor elements 1426, 1428 can be positionally secured with respect to one another. Alternatively, the body tube can be configured as a telescopic tube or otherwise adapted to permit adjustability between anchor elements 1426, 1428 to accommodate patients having abdominal walls of smaller or larger thicknesses, which may be particularly important in bariatric surgical procedures. For that reason, one or more anchor elements 1426, 1428 can alternatively be slideable to permit secure anchoring.

As embodied, the surgical access device 1400 includes a unitary housing 110 and body tube 120, but it is to be understood that a separable configuration can be effected as with above-described embodiments, and vice versa. In FIG. 16, a surgical instrument is illustrated extending through a central device port 111 and through the lumen 1601 of the surgical access device 1400. Further the access port is also shown as being stabilized by suture tethers 971, attached to anchor points 119 provided on the housing 110.

In the illustrated embodiment, the anchor elements 1426, 1428 are substantially annular in configuration and are inflatable by introduction of insufflation gas by way of a multi-way valve 1429, fill tube 1424 and one or more internal fill channels 1629 (See FIG. 16). The configuration of the anchor elements 1426, 1428, if provided, can vary and is not limited to the illustrated configuration. The anchor elements 1426, 1428 can be inflatable with a fluid such as insufflation gas, as described above, or alternatively with a benign liquid such as water or saline. Such liquid can be inserted and withdrawn by syringe through a self-sealing septum, for example. Further, the anchor elements can be passively compressible and expandable, as with a compressible foam material instead of providing inflation capability.

FIG. 17 is a top isometric view of a further embodiment of a surgical access device 1700 in accordance with the invention, having an engagement-promoting surface treatment 1722 provided on a body tube 1720 thereof. The engagement-promoting surface treatment 1722 is adapted to inhibit unintentional withdrawal of the surgical access device 1700 from its intended position, typically from the abdominal wall of the patient. The surface treatment 1722 can include, for example, textures such as circumferential ridges, or a high friction material such as a rubber coating.

In accordance with an alternate aspect of the invention, a surgical access device can configured with a linear path, or a path having another shape. In accordance with such aspects, the seal member, corresponding to seal member 214, is shaped to match with the moveable device port channel, such as channel 115, to maintain sealed access through the surgical access device. As mentioned above, the central device port 111 can be configured as a radially moveable device port in such a manner.

Devices in accordance with the invention are dimensioned similarly to other devices in the art, but sufficiently large to include all required elements. In accordance with one aspect, the body tube of surgical access devices of the invention has about a 2.5 centimeter outer diameter. The inner device ports, such as the stationary and moveable device ports, can be sized to accommodate a range of instrument diameters, such as instruments of about 5.0 mm in diameter to about 10.0 mm in diameter, but not limited to this range. The subject access devices can simultaneously include device ports of multiple sizes, as desired or required. Instruments that can be inserted through the subject access devices include any surgical instrument suitably sized, including but not limited to endoscopes, graspers, dissectors, staplers, and the like.

In accordance with a further aspect of the invention, a plurality of moveable device ports can be provided between inner and outer channel walls, as illustrated in the above embodiments. A surgical access device can include such moveable device ports, and be configured such that movement of one or more rotational elements, such as one or more rings held on the housing, effects arcuate translation of one or more moveable device ports along the channel. Such an embodiment can include one ring that simultaneously moves all moveable device ports, or alternatively, separate rings for each device port, for example. Such a configuration can be embodied as a planetary-type gear arrangement, with mutual frictional engagement, or alternatively gear teeth to enhance engagement and response to a call for movement.

Further, in accordance with another aspect, the invention further includes a method of accessing a surgical operative space, comprising the steps of forming an incision through an anatomical structure and inserting a surgical access device through the incision, where the surgical access device has a housing portion having an axial bore extending therethrough, defining a central axis of the surgical access device, and at least one moveable device port, moveable about an annular path corresponding to an annular seal member, each moveable device port defining a lumen extending therethrough, each lumen being substantially parallel to the central axis of the surgical access device. In accordance with this aspect, the operative space can be a patient's abdominal cavity and the anatomical structure can be the patient's abdominal wall.

The devices and related methods of the present invention, as described above and shown in the drawings, provide for surgical access devices with superior properties including enhanced control and reduced interference from surgical instruments being used simultaneously during a surgical procedure, particularly single-incision procedures.

It will be apparent to those skilled in the art that various modifications and variations can be made to the devices, and related methods of the present invention without departing from the spirit or scope of the invention. Thus, it is intended that the present invention include modifications and variations that are within the scope of the appended claims and their equivalents.

What is claimed is:

1. A surgical access device for use in laparoscopic surgical procedures, comprising:
a) a housing portion having an axial bore extending therethrough, defining a central axis of the surgical access device; and
b) at least one moveable device port, moveable about an annular path corresponding to an annular seal member, each moveable device port defining a lumen extending therethrough, each lumen being substantially parallel to the central axis of the surgical access device.

2. The surgical access device of claim 1, wherein the axis device includes a substantially axially central stationary device port held in the housing portion, the stationary device port being restrained from lateral and axial movement, relative to the central axis.

3. The surgical access device of claim 2, wherein the stationary device port is rotatable about a central axis thereof.

4. The surgical access device of claim 1, wherein the moveable device port includes a seal member held within a moveable housing, the moveable housing being configured to engage the annular path.

5. The surgical access device of claim 4, wherein the seal member of the moveable device port includes a duckbill-type sealing element configured to seal the lumen of the moveable device port in the absence of an instrument inserted therethrough.

6. The surgical access device of claim 5, wherein the seal member of the moveable device port includes an annular seal configured to sealably engage an outer surface of an instrument when inserted therethrough.

7. The surgical access device of claim 1, wherein a plurality of moveable device ports are provided.

8. The surgical access device of claim 1, wherein three moveable device ports are provided.

9. The surgical access device of claim 1, wherein the circular path is an annular channel defined by the housing.

10. The surgical access device of claim 9, wherein the annular channel restrains the moveable device port from moving in any direction other than along the circular path.

11. The surgical access device of claim 10, wherein the annular channel is defined within the housing by radially outer and inner channel walls and axially upper and lower channel walls.

12. The surgical access device of claim 11, wherein the axially upper and lower channel walls only partially extend radially toward a centerline of the annular channel, permitting passage of instruments through a moveable device port lumen and through the upper and lower channel walls.

13. The surgical access device of claim 12, wherein the annular channel includes a plate formed of a lubricious material on the axially lower channel wall, facilitating movement of the at least one moveable device port.

14. The surgical access device of claim 13, wherein the annular seal member is compressed by and at least partly positionally secured by the plate.

15. The surgical access device of claim 1, wherein the annular seal member is includes two sealing portions extending toward one another about the annular path, and resiliently mutually contacting one another to inhibit flow of insufflation gas though the seal member.

16. The surgical access device of claim 1, further comprising a body tube extending from a lower end of the housing portion, the body tube defining a central lumen extending axially therethrough.

17. The surgical access device of claim 16, wherein at least one engagement feature is provided on the body tube to facilitate engagement of the surgical access device with an anatomical structure of a patient.

18. The surgical access device of claim 17, wherein the anatomical structure is a portion of the abdominal wall of the patient.

19. The surgical access device of claim 18, wherein the at least one engagement feature is a substantially annular engagement member extending radially outwardly from an outer circumferential wall of the body tube.

20. The surgical access device of claim 19, wherein the annular engagement member is inflatable with a fluid to expand the volume of the engagement member to promote engagement with the anatomical structure.

21. The surgical access device of claim 20, wherein the annular engagement member is in communication with an insufflation connection on the surgical access device.

22. The surgical access device of claim 21, wherein a multi-way valve is provided interposed between the insufflation connection, an insufflation output into the surgical access device and into at least one engagement member.

23. The surgical access device of claim 16, further comprising an insufflation port in fluid communication with the lumen, arranged below the annular seal member, adapted and configured for providing insufflation gas to an operative cavity.

24. The surgical access device of claim 1, further comprising at least one anchor point on the housing portion, configured and adapted for engagement with a suture material for tethering to an anatomical structure of a patient.

25. The surgical access device of claim 24, wherein the anatomical structure is a portion of the abdominal wall of the patient.

26. A method of accessing a surgical operative space, comprising the steps of:
a) forming an incision through an anatomical structure; and
b) inserting a surgical access device through the incision, the surgical access device having:
i) a housing portion having an axial bore extending therethrough, defining a central axis of the surgical access device; and
ii) at least one moveable device port, moveable about an annular path corresponding to an annular seal member, each moveable device port defining a lumen extending therethrough, each lumen being substantially parallel to the central axis of the surgical access device.

27. The method of claim 26, wherein the operative space is a patient's abdominal cavity and the anatomical structure is the patient's abdominal wall.
